(19) United States
(12) Patent Application Publication
(10) Pub. No.: US 2022/0304728 A1
(43) Pub. Date: Sep. 29, 2022

(54) INTERNAL ELASTIC BRACE FOR TREATING SCOLIOSIS

(71) Applicant: Grant D. Hogue, San Antonio, TX (US)

(72) Inventor: Grant D. Hogue, San Antonio, TX (US)

(21) Appl. No.: 17/607,955

(22) PCT Filed: May 1, 2020
(86) PCT No.: PCT/US2020/030978

§ 371 Date: Nov. 1, 2021

Related U.S. Application Data
(60) Provisional application No. 62/841,691, filed on May 1, 2019.

(51) Int. Cl. A61B 17/70

(52) U.S. Cl. CPC A61B 17/707; A61B 2017/564

(57) ABSTRACT

Certain embodiments are directed to a scoliosis treatment device, an internal elastic brace (IEB) for scoliosis, and methods of using or deploying the same to treat scoliosis. The scoliosis treatment device includes various components that are assembled and deployed to provide a compressive force to the convex side of an abnormally curved spine by tethering the ribs on the convex side of the spinal curvature.

### RELATED APPLICATIONS

This application claims priority to U.S. Provisional Application Ser. No. 62/841,691 filed May 1, 2019, which is incorporated herein by reference in its entirety.

### STATEMENT REGARDING FEDERALLY FUNDED RESEARCH

None.

### I. FIELD

Certain embodiments are directed to the field of medical devices, and methods that deploy such devices, to correct spinal deformities, in particular spinal scoliosis.

### II. BACKGROUND

Scoliosis is a sideways curvature of the spine that occurs most often during the growth spurt just before puberty. While scoliosis can be caused by conditions such as cerebral palsy and muscular dystrophy, the cause of most scoliosis is unknown.

Most cases of scoliosis are mild, but some children develop spine deformities that continue to get more severe as they grow. Severe scoliosis can be disabling. An especially severe spinal curve can reduce the amount of space within the chest, making it difficult for the lungs to function properly.

Traditionally, scoliosis is treated by a spinal fusion performed posteriorly. Spinal fusion involves techniques designed to mimic the normal healing process of broken bones. During spinal fusion, a surgeon places bone or a bonelike material within the space between two spinal vertebrae. Metal plates, screws and rods may be used to hold the vertebrae together, so they can heal into one solid unit. Because spinal fusion surgery immobilizes parts of the spine, it changes the way the spine can move. This places additional stress and strain on the vertebrae above and below the fused portion, and may increase the rate at which those areas of the spine degenerate.

Other treatment methods include braces and Schroth exercise routines for intervention in scoliosis. Further, braces and the German-developed Schroth exercises have been used separately and together to correct various types of scoliosis. Some conditions cannot be fully corrected, and if they progress may necessitate spinal surgery procedures, such as those described above, including fusion and/or the implantation of medical devices to internally support the spine in a correct position. Hence, it is desirable to provide corrective methods to patients to avoid future surgical intervention which poses risks, expense and at least temporary absence of the patient from work and/or school.

Others (U.S. Pat. No. 5,261,908) have attempted to utilize a device originally designed to treat thoracic deficiency (Expandable Vertical Prosthetic Rib) for the treatment of scoliosis; however, the rigidity of the device and the attachment mechanism degraded the integrity of the rib rendering this device unsuitable for treating scoliosis.

There remains a need for additional devices and methods for treating scoliosis.

### SUMMARY

Certain embodiments are directed to a scoliosis treatment device, an internal elastic brace (IEB) for scoliosis, and methods of using or deploying the same to treat scoliosis. The scoliosis treatment device includes various components that are assembled and deployed to provide a compressive force to the convex side of an abnormally curved spine by tethering the ribs on the convex side of the spinal curvature. A first end of the device will be anchored to a first rib and the second end of the device will be anchored to a second rib by a first and second anchor respectively, with the first and second anchor being attached through an intervening segment or tether configured to tether the first and second rib providing a compressive force to the spine or vertebrae located with the abnormal curvature. The length of the intervening segment being selected to apply a compressive force to the spine without causing injury to ribs or the rib cage of a subject. The compressive force may be established upon deployment of the device, during growth of the subject or both at deployment and during growth of the subject. The intervening segment or tether can be an elastic or dynamic tether. As used herein, the term “elastic” refers to the ability of a material to deform (e.g., stretch or bend) and the deformed material to return to its initial state after a deforming load is removed. In a related aspect, it will be understood by those skilled in the art that the term “inelastic” refers to material that resists stretching and elongation (e.g., metals and alloys). In certain aspects the first and second anchor, and the intervening segment or tether are attached during deployment and remain attached until removed. The anchors are configured to attach to a subject's rib and to the intervening segment or tether forming an elastic brace. The anchors fix the ends of the intervening segment to the ribs in such a way so as to provide a compression force on the convex portion of the spine during the growth of the individual by fixing the respective position of the first and second rib and/or providing an appropriate force pulling the ribs towards each other. While not being bound by theory, the device provides compression to one side of the spine via the ribs so that the patient essentially grows straight based on the Hueter-Volkmann Principle (during skeletal immaturity growth is slowed by compression and accelerated by tension).

The anchor is configured to be position on the rib on the side distal to the position of the tether and the other rib involved in the deployment of the device. The anchor can be a curved plate that conforms to the curvature of the rib. In certain aspects the anchor can be fixed to the rib using two or more locked screws. In other aspects, the anchor is held in place by the force applied by the tether. The anchor is configured to receive and attach to one end of the tether that is drawn through a path made by a surgeon through the rib or the anchor has a projection (e.g., a rod or pin) that is configured to extend through a path made in the rib to engage and attach to the tether opposite the rib from the anchor. The interface between the tether and the anchor can be secured by a clamp(s), staple(s), pin(s), loop or the like.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps), but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Illustration of the IEB deployed and in operation.

FIG. 2. Illustration of the anchor and its interface with an intervening segment or tether.

### DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Embodiments of the IEB described herein is safer than traditional posterior spinal fusion. The device can be deployed as a day surgery without requiring a full service hospital. IN certain instance, IEB deployment does not require entry into the chest cavity (unlike vertebral body tethering) and requires a less demanding surgical skill-set. Furthermore, the device does not fuse the spine and is reversible.

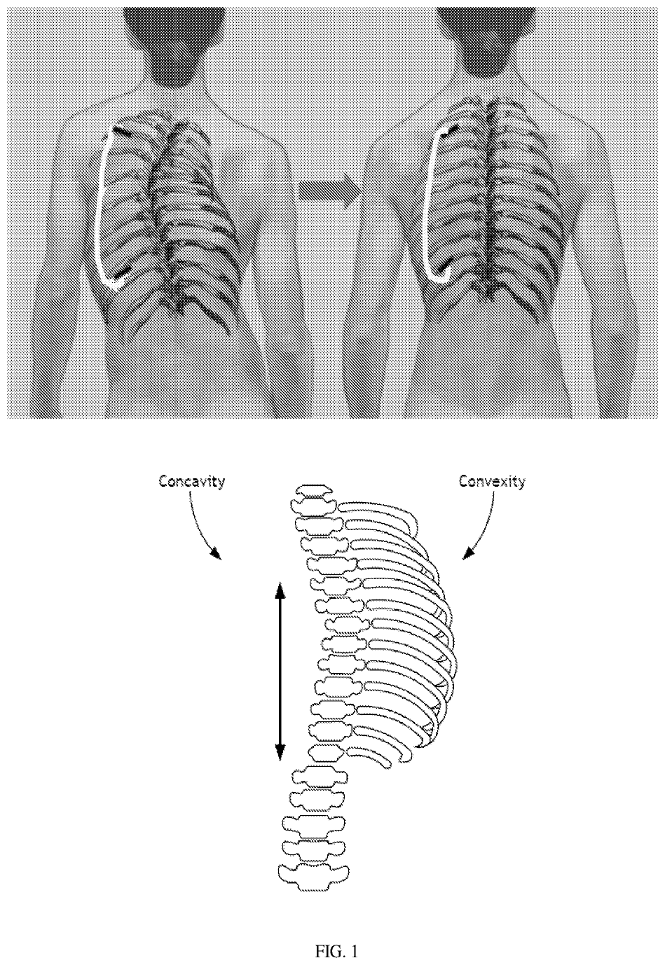

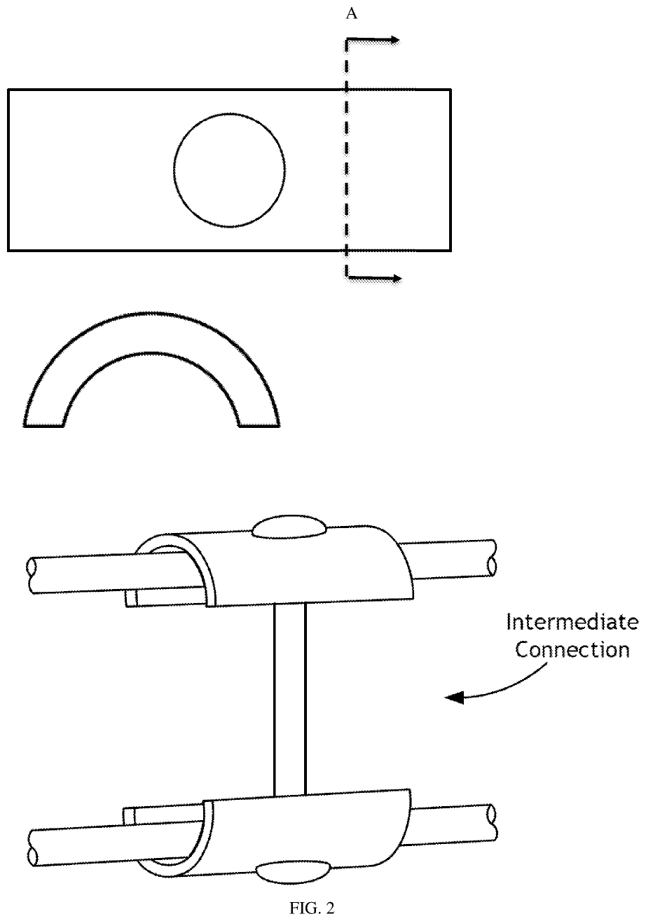

FIG. 1 illustrates the bracing of the convex side of a spinal curvature and the straightening of the curvature via growth modulation over time. FIG. 2 is a more detailed view of one embodiment of an internal elastic brace (IEB), illustrating an example of one embodiment of a rib attachment anchor (overhead and side view) and an assembly of the anchor with a tether component deployed in a therapeutic situation.

Anchor Portion or Component. An anchor is configured to attach to a subject's rib and to the intervening segment or tether forming an elastic brace. The anchors fix the ends of the intervening segment to the ribs in such a way so as to provide a compression force pulling the ribs towards each other or maintaining the initial distance between ribs. The anchor is configured to be positioned on the rib on the side distal to the position of the tether and the other rib involved in the deployment of the device. The anchor can be a curved plate that conforms to the curvature of the rib. In certain aspects the anchor can be configured to receive two or more screws for attachment to the rib. In other aspects, the anchor can have bone anchors on the face of the anchor to interact and stabilize its position on the rib. In other aspects, the anchor is held in place by the force applied by the tether. The anchor is configured to receive and attach to one end of the tether. In certain aspects, the anchor provide a hole or slot through which the end portion of a tether is positioned. After being positioned the end of the tether is knotted or capped or a button or stop deployed so that the tether does not pass back through anchor.

The anchor can be a rigid plate. The anchor can be in the shape of a curved rectangle or other planar shape with a thickness of 0.01 to 0.5 cm, a width sufficient to wrap or complement the convex curvature of the rib (e.g., at least, at most, or about 0.5, 1, 1.5, 2.0, 2.5, 3.0, 3.5, 4.0, 4.5, 5.0 cm, including all values and ranges there between), and a length of at least, at most, or about 1, 2, 3, 4, 5, 6, 7, 8, 9, 10 cm, including all values and ranges there between. The anchor can be made of a variety of materials. In certain aspects, the anchor can be metal; metal alloy; carbon fiber; synthetic resins such as polysulphones, polyphenyl sulphones, epoxides, acrylic resins, polyolefins, polyoxymethylene, polyphenylene sulphide, polyetherketones, polyamides and polyesters, preferably reinforced with carbon, glass and/or other high-strength synthetic fibers.

Intervening Segment or Tether. The intervening segment or tether can be an elongated segment having a circular, oval, or rectangular cross section. The tether have a thickness or diameter of at least, at most, or about 0.1, 0.2, 0.3, 0.4, 0.5, 0.6, 0.7, 0.8, 0.9, 1.0 cm, including all values and ranges there between; a width of at least, at most, or about 0.5, 1, 1.5, 2.0, 2.5, 3.0, 3.5, 4.0, 4.5, 5.0 cm, including all values and ranges there between; and a length (when deployed) of at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 cm, including all values and ranges there between. In certain aspects, the tether can be provided or made at a pre-deployment length that can be cut or adjusted by the personnel deploying the IEB device to an appropriate length for a particular subject. The tether can be from suitable elastic materials include, but are not limited to, reinforced tendon graft, polyvinylchloride, polyurethane, polyethylene, nylon, silicone, fluoropolymers, polypropylene, polyesters, FIBERWIRE®, mersilene tape, or the like. In other aspects, the tether can include braided filaments of ultrahigh molecular weight polyethylene (UHMWPE) or similar substance, braided with strands of polyester, collagen, or other suture materials, such as PET, PEEK, silk nylon, and absorbable polymers, among many others. In certain instance the material will have a Young's modulus of elasticity of between 6 and 11.

1. A kit for assembly of an internal elastic brace for the treatment of scoliosis comprising:
(a) a first and second rib attachment anchor; and
(b) a rib attachment tether,
wherein the components are configure for a first end of the rib attachment tether to be anchored to a first rib by a first rib attachment anchor and the second end of the rib attachment tether to be anchored to a second rib by a second rib attachment anchor to tether the first and second rib providing a compressive force to the spine or vertebrae located with the abnormal curvature.

2. A method of deploying the kit of claim 1 comprising:
(a) forming a lumen in a first and second rib;
(b) positioning a first end of a rib tether through the lumen in a first rib;
(c) securing the rib tether to a first rib attachment anchor;
(d) positing a second end of the rib tether through the lumen in a second rib; and
(e) securing the rib tether to a second rib attachment anchor.

3. The method of claim 2, wherein the first second rib are non-adjacent ribs.
